(19) United States
(12) Patent Application Publication
(10) Pub. No.: US 2002/0068365 A1
(43) Pub. Date: Jun. 6, 2002

(54) CONTROLLED RELEASE NITRIC OXIDE PRODUCING AGENTS

(76) Inventor: ERIC H. KUHRTS, REDWOOD CITY, CA (US)

Correspondence Address: BIRCH STEWART KOLASCH & BIRCH, PO BOX 747, FALLS CHURCH, VA, 22040-0747, US

(21) Appl. No.: 09/123,849

(22) Filed: Jul. 28, 1998

(51) Int. Cl. G01N 33/48; G01N 33/566

(52) U.S. Cl. 436/501; 436/63

(57) ABSTRACT

Disclosed are various controlled release pharmaceutical compositions that include an agent that enhances or modulates the endogenous production of nitric oxide in a mammal. Controlled release pharmaceutical compositions of L-arginine, its salts, peptides, and biological equivalents, together with methods of using the compositions are included. Also included are controlled release pharmaceutical compositions of botanical extracts that modulate or enhance the production of nitric oxide, either alone or in combination with L-arginine or its biological equivalent.

### BACKGROUND OF THE INVENTION

### Field of the Invention

This invention relates to controlled release compositions containing nitric oxide enhancing or modulating agents, more particularly to controlled release compositions containing L-arginine, L-citrulline, L-ornithine, and their salts, complexes, or peptides, as well as botanical substances and extracts such as ginkgo biloba, bioflavonoids, and garlic for pharmaceutical uses.

Nitric oxide (NO) plays an important role in the regulation of many physiological functions such as vasodilatation, atherosclerosis, platelet aggregation, restenosis, hypertension, reperfusion injury, renal failure, and erectile dysfunction (Ignarro LJ. Physiological Significance of Endogenous Nitric Oxide. Seminars in Perinatology, 1991; Vol. 15, 1; 20-26). Endogenous NO is synthesized by different isoforms of the enzyme nitric oxide synthase (NOS) from the amino acid L-arginine. (Moncada S, Higgs E A. The L-arginine-nitric oxide pathway (N England J Med 1993: 329:2002-2012). NOS is a cytochrome p450 protein enzyme which requires certain cofactors. The biosynthesis of endogenous NO from L-arginine by NOS involves the basic guanidino nitrogen atoms of L-arginine, and the intermediate product is L-citrulline.

The liver contains enzymes that convert drugs and other dietary chemicals to metabolites which can then be more easily eliminated by the body in the urine and the feces. This conversion process or biotransformation of the drug or therapeutic compound may, in many cases, influence the duration of action or the intensity (pharmacodynamics) of the compound. The rate of metabolism and the extent of metabolism can have a profound effect on the therapeutic parameters of the drug, which in turn is a reflection of the bioavailability.

Because of metabolism issues, many drugs or natural therapeutic agents such as L-arginine must be taken numerous times a day to achieve the desired pharmacological effects.

Cytochrome p450 is one of the many pharmaceutical-metabolizing enzyme systems of the liver, but is perhaps the enzyme system that plays the most important role in determining the rate of elimination of drugs. Each of the various enzyme systems in the liver is comprised of many individual enzymes, each of which is capable of metabolizing a wide variety of therapeutic substances or chemicals. The cytochrome P450 system in the liver consists of at least ten individual P450 enzymes. The metabolism of therapeutic agents by cytochrome P450 often represents the rate-limiting step in pharmaceutical elimination. Therefore, factors that decrease the activity of P450 enzymes usually prolong the effects of drugs, whereas factors that increase cytochrome P450 activity have the opposite effect.

Since the conversion of L-arginine to NO is a metabolic process involving cytochrome P450 (Sessa WC, The nitric oxide synthase family of proteins; J Vasc Res 1994; 31:131-143), the rate of presentation of L-arginine to the liver can effect its conversion to NO via cytochrome P450 metabolism. Furthermore, by prolonging and slowing the transit of a solid dosage form such as a tablet, granules, or coated particles through the window of absorption with a sustained-release formulation, metabolism of the therapeutic agent can be effected. Cytochrome P450 enzymes are also located in the gastrointestinal tract, so slowing down the rate of presentation and exposure of the drug or therapeutic agent to these enzymes should effect their metabolism. Therapeutic agents that are subject to first pass metabolism via the portal vein, and are presented to the liver prior to systemic circulation, may be influenced more profoundly by incorporation in sustained-release dosage forms that slow transit through the small intestine. In this way, the rate and extent of metabolism may be effected.

L-arginine, L-ornithine, arginine silicate, salts of L-arginine, complexes, and peptides of L-arginine are preferred substrates for the endogenous production of NO. Unfortunately, fairly large doses (3 to 10 grams per dose) of L-arginine are required to enhance NO production, and single doses in excess of a few grams are inadequately absorbed because they result in diarrhea (bowel intolerance) due to the very basic nature of the amino acid, and saturation of absorption systems. L-arginine free base, which gram for gram yields the most arginine for substrate production of NO, has a pH range of 10.5-12.0, and is extremely alkaline. Oral consumption of a single dose of 3 grams or more of L-arginine free base results in bowel intolerance within a few hours in the majority of subjects, which significantly reduces the amount of arginine that is absorbed. Diarrhea generally manifests as intestinal hypermotility and rapid transport, speeding up gastric emptying and shortening transit time for solutes in the window of absorption. Controlled-release formulations of L-arginine modulate the exposure of the alkaline amino acid to the gastrointestinal tract, and reduce the concentration of the amino acid to the extent that greater absorption is possible due to reduced bowel intolerance. In addition, the saturation or overwhelming of absorption systems can be avoided. In this way less L-arginine is lost to diarrhea, and more is absorbed for production of nitric oxide.

All of the studies conducted with L-arginine that relate to the benefits of NO production have either involved intravenous administration or oral administration of immediate-release formulations in repeated doses throughout the day. For example, in the study “Effect of Supplemental Oral L-Arginine on Exercise Capacity in Patients With Stable Angina Pectoris” by Ceremuzynski et al; American Journal of Cardiology; 1997,80 (3); 331-3, the subjects were given two 1 gram capsules (2 grams) 3 times a day, at 9 A.M., 2 P.M., and 10 P.M. An example of the intravenous administration of L-arginine can be found in “L-Arginine Infusion Decreases Platelet Aggregation Through An Intraplatelet Nitric Oxide Release”; Marietta et al; Thrombosis Research; 1997; 88, (2): 229-35. In that study subjects were given 30 grams of L-arginine as an infusion. This raised circulating levels of L-arginine up to 100 fold compared to baseline levels. This same dose would have been impossible to administer orally as it would not be tolerated by the gastrointestinal tract.

These represent undesirable routes of administration for a variety of reasons. First of all, intravenous administration remains undesirable because of the expense and difficulty involved in administering such medications intravenously. Subjects will always prefer oral administration over injection or infusion, as it avoids painful insertion of needles. Additionally, there is the enhanced danger of infection. Intravenous administration also involves a clinic and a medical professional, and is not suitable or practical for daily usage.

Oral administration, while desirable, represents problems in that administration of the compound in conventional oral dosage forms at levels necessary to generate nitric oxide results in diarrhea, thus significantly reducing the bioavailability of the compound. Consequently, despite the usefulness of L-arginine and its biological equivalents in treating a variety of medical conditions, there remains no good dosage form for administering L-arginine in the quantities necessary for generation of significant pharmacological amounts of nitric oxide. There is therefore a need for improved dosage forms of L-arginine and its biological equivalents for use in oral administration.

Furthermore, certain botanical extracts such as the bioflavonoids have a modulating or regulating effect on nitric oxide production. By combining these substances with substrate agents such as L-arginine, more effective control over nitric oxide production is possible. For example, French maritime pine bark extract, a mixture of bioflavonoids, is known to modulate nitric oxide metabolism in inflammation. Ginkgo biloba and garlic are also known to regulate nitric oxide metabolism. Controlled release formulations of these botanical extracts would enable more control over NO modulation.

### SUMMARY OF THE INVENTION

In one aspect, the invention relates to a controlled release pharmaceutical composition comprising a nitric oxide stimulating agent In another aspect, the invention relates to a composition comprising L-arginine or L-ornithine, their biological equivalents, as salts, complexes, or peptides in controlled-release formulations to be delivered orally.

### DETAILED DESCRIPTION OF THE INVENTION

This invention relates to the discovery that the bioavailability of L-arginine and its biological equivalents can be enhanced through incorporation into a controlled release oral dosage form. This incorporation provides higher absorption of L-arginine, thus increasing L-arginine's effect.

If L-arginine is incorporated as a salt, salt formers that may, for example, be used are conventional bases or cations which are physiologically acceptable in the salt form. Examples thereof are: alkali metals or alkaline earth metals, ammonium hydroxide, basic amino acids such as arginine and lysine, amines of formula NR₁R₂R₃ where the radicals R₁, R₂ and R₃ are the same or different and represent hydrogen, C₁-C₄-alkyl or C₁-C₄ oxyalkyl such as mono- and diethanol-amine, 1-amino-2-propanol, 3-amino-1-propanol; alkylene diamines having one alkylene chain composed of 2 to 6 carbon atoms such as ethylene diamine or hexamethylene tetramine, and saturated cyclic amino compounds with 4-6 cyclic carbon atoms such as piperidine, piperazine, pyrrolidine, morpholine; N-methyl glucamine, creatine, or tromethamine.

Should L-arginine be used in the form of its salts, the salt former may also be used in excess, i.e. in an amount greater than equimolar.

Additionally, L-arginine or L-ornithine or its biological equivalet may be taken to mean, within the context of the invention, to include various analogs, prodrugs, peptides, various oxidation states of the fundamental L-arginine molecule, metabolites, and salts of any of the above. For example, included might be, a hydrochloride salt of L-arginine, or arginine silicate as described in U.S. Pat. No. 5,707,970. Such L-arginines may be administered to a mammal.

Controlled release within the scope of this invention can be taken to mean any one of a number of extended release dosage forms. The following terms may be considered to be substantially equivalent to controlled release, for the purposes of the present invention: continuous release, controlled release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slowing acting, sustained action, sustained-action medications, and extended release. Further discussions of these terms may be found in Lesczek Krowczynski, Extended-Release Dosage Forms, 1987 (CRC Press, Inc.).

The various controlled release technologies cover a very broad spectrum of drug dosage forms. Controlled release technologies include, but are not limited to physical systems and chemical systems. Physical systems include, but not limited to, reservoir systems with rate-controlling membranes, such as microencapsulation, macroencapsulation, and membrane systems; reservoir systems without rate-controlling membranes, such as hollow fibers, ultra microporous cellulose triacetate, and porous polymeric substrates and foams; monolithic systems, including those systems physically dissolved in non-porous, polymeric, or elastomeric matrices (e.g., non-erodible, erodible, environmental agent ingression, and degradable), and materials physically dispersed in non-porous, polymeric, or elastomeric matrices (e.g., non-erodible, erodible, environmental agent ingression, and degradable); laminated structures, including reservoir layers chemically similar or dissimilar to outer control layers; and other physical methods, such as osmotic pumps, or adsorption onto ion-exchange resins.

Chemical systems include, but are not limited to, chemical erosion of polymer matrices (e.g., heterogeneous, or homogeneous erosion), or biological erosion of a polymer matrix (e.g., heterogeneous, or homogeneous).

Hydrogels may also be employed as described in “Controlled Release Systems: Fabrication Technology”, Vol. 11, Chapter 3; p 41-60; “Gels For Drug Delivery”, Edited By Hsieh, D.

Controlled release drug delivery systems may also be categorized under their basic technology areas, including, but not limited to, rate-preprogrammed drug delivery systems, activation-modulated drug delivery systems, feedback-regulated drug delivery systems, and site-targeting drug delivery systems.

In rate-preprogrammed drug delivery systems, release of drug molecules from the delivery systems “preprogrammed” at specific rate profiles. This may be accomplished by system design, which controls the molecular diffusion of drug molecules in and/or across the barrier medium within or surrounding the delivery system.

In activation-modulated drug delivery systems, release of drug molecules from the delivery systems is activated by some physical, chemical or biochemical processes and/or facilitated by the energy supplied externally. The rate of drug release is then controlled by regulating the process applied, or energy input.

In feedback-regulated drug delivery systems, release of drug molecules from the delivery systems may be activated by a triggering event, such as a biochemical substance, in the body. The rate of drug release is then controlled by the concentration of triggering agent detected by a sensor in the feedback regulated mechanism.

In a site-targeting controlled-release drug delivery system, the drug delivery system targets the active molecule to a specific site or target tissue or cell. This may be accomplished, for example, by a conjugate including a site specific targeting moiety that leads the drug delivery system to the vicinity of a target tissue (or cell), a solubilizer that enables the drug delivery system to be transported to and preferentially taken up by a target tissue, and a drug moiety that is covalently bonded to the polymer backbone through a spacer and contains a cleavable group that can be cleaved only by a specific enzyme at the target tissue.

While a preferable mode of controlled release drug delivery will be oral, other modes of delivery of controlled release compositions according to this invention may be used. These include mucosal delivery, nasal delivery, ocular delivery, transdermal delivery, parenteral controlled release delivery, vaginal delivery, rectal delivery, and intrauterine delivery.

There are a number of controlled release drug formulations that are developed preferably for oral administration. These include, but are not limited to, osmotic pressure-controlled gastrointestinal delivery systems; hydrodynamic pressure-controlled gastrointestinal delivery systems; membrane permeation-controlled gastrointestinal delivery systems, which include microporous membrane permeation-controlled gastrointestinal delivery devices; gastric fluid-resistant intestine targeted controlled-release gastrointestinal delivery devices; gel diffusion-controlled gastrointestinal delivery systems; and ion-exchange-controlled gastrointestinal delivery systems, which include cationic and anionic drugs.

Enteric coatings may be applied to tablets to prevent the release of drugs in the stomach either to reduce the risk of unpleasant side effects or to maintain the stability of the drug which might otherwise be subject to degradation of expose to the gastric environment. Most polymers that are used for this purpose are polyacids that function by virtue or the fact that their solubility in aqueous medium is pH-dependent, and they require conditions with a pH higher then normally encountered in the stomach.

Enteric coatings may be used to coat a solid or liquid dosage form of the NO enhancing agent. For example, enteric coatings promote the L-arginine remaining physically incorporated in the dosage form for a specified period when exposed to gastric juice. Instead, the enteric coatings are designed to disintegrate in the higher pH of the intestinal fluid for ready absorption. Delay of the L-arginine absorption is dependent on the rate of transfer through the gastrointestinal tract, and so the rate of gastric emptying is an important factor. Some investigators have reported that a multiple-unit type dosage form, such as granules, may be superior to a single-unit type. Therefore, in a preferable embodiment, the L-arginine may be contained in an enterically coated multiple-unit dosage form. In a more preferable embodiment, the L-arginine dosage form is prepared by spray-coating granules of L-arginine with an enteric coating agent solid dispersion on an inert core material. These granules can result in prolonged absorption of the drug with good bioavailability.

Typical enteric coating agents include, but are not limited to, hydroxypropylmethylcellulose phthalate, methacrylic acid-methacrylic acid ester copolymer, polyvinyl acetate-phthalate and cellulose acetate phthalate. Various enteric coating materials may be selected on the basis of testing to achieve an enteric coated dosage form designed ab initio to have a preferable combination of dissolution time, coating thicknesses and diametrical crushing strength. (see for example “Aqueous Polymeric Coatings For Pharmaceutical Dosage Forms, edited by James W. McGinity, Marcel Dekker, New York, 1989)

On occasion, the performance of an enteric coating may hinge on its permeability. With such oral drug delivery systems, the drug release process may be initiated by diffusion of aqueous fluids across the enteric coating. Investigations have suggested osmotic driven/rupturing affects as important release mechanisms from enteric coated dosage forms.

Combinations of coating agents may also be incorporated such as ethylcellulose and hydroxypropylmethylcellulose, which can be mixed together and sprayed onto the L-arginine in a fluid bed granulator.

Another type of useful oral controlled release structure is a solid dispersion. A solid dispersion may be defined as a dispersion of one or more active ingredients in an inert carrier or matrix in the solid state prepared by the melting (fusion), solvent, or melting-solvent method. The solid dispersions may be also called solid-state dispersions. The term “coprecipitates” may also be used to refer to those preparations obtained by the solvent methods.

Solid dispersions may be used to improve the solubilities and/or dissolution rates of poorly water-soluble forms of L-arginine such as the free base. The solid dispersion method was originally used to enhance the dissolution rate of slightly water-soluble medicines by dispersing the medicines into water-soluble carriers such as polyethylene glycol or polyvinylpyrrolidone,

The selection of the carrier may have an influence on the dissolution characteristics of the dispersed drug because the dissolution rate of a component from a surface may be affected by other components in a multiple component mixture. For example, a water-soluble carrier may result in a fast release of the drug from the matrix, or a poorly soluble or insoluble carrier may lead to a slower release of the drug from the matrix.

Aqueous dispersions may also be formulated. Of particular interest for L-arginine aqueous dispersions are polymeric hydroabsorptive agents such as hydrcolloid fibers, which will help to absorb water in the gastrointestinal tract, helping to minimize the potential for diarrhea, while also providing sustained-release.

Examples of carriers useful in solid and aqueous dispersions according to the invention include, but are not limited to, water-soluble polymers such as guar gum, glucommannan, psyllium, gum acacia, polyethylene glycol, polyvinylpyrrolidone, hydroxypropyl methylcellulose, and other cellulose ethers such as methylcellulose, and sodium carboxymethylcellulose. Powdered drink mixes which are designed to be added to water or other liquids incorporating microspheres of sustained-release L-arginine with a hydrocolloid polymer such as those previously listed are also suitable.

There are various methods commonly known for preparing solid dispersions. These include, but are not limited to the melting method, the solvent method and the melting-solvent method.

In the melting method, the physical mixture of a drug in a water-soluble carrier is heated directly until it melts. The melted mixture is then cooled and solidified rapidly while rigorously stirred. The final solid mass is crushed, pulverized and sieved. Using this method a super saturation of a solute or drug in a system can often be obtained by quenching the melt rapidly from a high temperature. Under such conditions, the solute molecule may be arrested in solvent matrix by the instantaneous solidification process. A disadvantage is that many substances, either drugs or carriers, may decompose or evaporate during the fusion process at high temperatures. However, this evaporation problem may be avoided if the physical mixture is heated in a sealed container. Melting under a vacuum or blanket of an inert gas such as nitrogen may be employed to prevent oxidation of the drug or carrier.

The solvent method has been used in the preparation of solid solutions or mixed crystals of organic or inorganic compounds. Solvent method dispersions may prepared by dissolving a physical mixture of two solid components in a common solvent, followed by evaporation of the solvent The main advantage of the solvent method is that thermal decomposition of drugs or carriers may be prevented because of the low temperature required for the evaporation of organic solvents. However, some disadvantages associated with this method are the higher cost of preparation, the difficulty in completely removing liquid solvent, the possible adverse effect of its supposedly negligible amount of the solvent on the chemical stability of the drug.

Another controlled release dosage form is a complex between an ion exchange resin and L-arginine equivalents. Ion exchange resin-drug complexes have been used to formulate sustained-release products of acidic and basic drugs. In one preferable embodiment, a polymeric film coating is provided to the ion exchange resin-drug complex particles, making drug release from these particles diffusion controlled.

Furthermore, compositions of L-arginine and biological equivalents according to the invention may be administered or coadministered with conventional pharmaceutical binders, excipients and additives. Many of these are controlled-release polymers which can be used in sufficient quantities to produce a sustained-release effect. These include, but are not limited to, gelatin, natural sugars such as raw sugar or lactose, lecithin, mucilage, plant gums, pectin's or pectin derivatives, algal polysaccharides, glucomannan, agar and lignin, guar gum, locust bean gum, acacia gum, xanthan gum, carrageenan gum, karaya gum, tragacanth gum, ghatti gum, starches (for example corn starch or amylose), dextran, polyvinyl pyrrolidone, polyvinyl acetate, gum arabic, alginic acid, tylose, talcum, lycopodium, silica gel (for example colloidal), cellulose and cellulose derivatives (for example cellulose ethers, cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate, cross-linked sodium carboxymethylcellulose, cross-linked hydroxypropylcellulose, high-molecular weight hydroxymethylpropycellulose, carboxymethyl-cellulose, low-molecular weight hydroxypropylmethylcellulose medium-viscosity hydroxypropylmethylcellulose hydroxyethylcellulose, hydroxypropylcellulose, hydroxypropylmethylcellulose, sodium carboxymethylcelulose, alkylcelluloses, ethyl cellulose, cellulose acetate, cellulose propionate (lower, medium or higher molecular weight), cellulose acetate propionate, cellulose acetate butyrate, cellulose triacetate, methyl cellulose, hydroxypropyl cellulose, or hydroxypropylmethyl cellulose), fatty acids as well as magnesium, calcium or aluminum salts of fatty acids with 12 to 22 carbon atoms, in particular saturated (for example stearates such as magnesium stearate), polycarboxylic acids, emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈J₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C₁-C₁₂-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other substances that may be used include: cross-linked polyvinyl pyrrolidone, carboxymethylamide, potassium methacrylatedivinylbenzene copolymer, high-molecular weight polyvinylacohols, low-molecular weight polyvinylalcohols, medium-viscosity polyvinylalcohols, polyoxyethyleneglycols, non-cross linked polyvinylpyrrolidone, polyethylene glycol, sodium alginate, galactomannone, carboxypolymethylene, sodium carboxymethyl starch, sodium carboxymethyl cellulose or microcrystalline cellulose; polymerizates as well as copolymerizates of acrylic acid and/or methacrylic acid and/or their esters, such as, but not limited to poly(methyl methacrylate), poly(ethyl methacrylate), poly(butyl methacylate), poly (isobutyl methacrylate), poly(hexyl methacrylate), poly (isodecyl methacrylate), poly(lauryl methacrylate), poly(phenyl methacrylate), poly(methyl acrylate), poly(isopropyl acrylate), poly(isobutyl acrylate), or poly(octadecyl acrylate); copolymerizates of acrylic and methacrylic acid esters with a lower ammonium group content (for example Eudragit® RS, available from Rohm, Somerset, N.J. ), copolymerizates of acrylic and methacrylic acid esters and trimethyl ammonium methacrylate (for example Eudragit® RL, available from Rohm, Somerset, N.J.); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethylacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine; poly (ethylene), poly (ethylene) low density, poly (ethylene) high density, poly (propylene), poly (ethylene oxide), poly (ethylene terephthalate), poly (vinyl isobutyl ether), poly (vinyl chloride) or polyurethane. Mixtures of any of the substances or materials listed herein may also be used in the practice of the invention.

Plasticizing agents that may be considered as coating substances useful are: Citric and tartaric acid esters (acetyl-triethyl citrate, acetyl tributyl-, tributyl-, triethyl-citrate); glycerol and glycerol esters (glycerol diacetate, - triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

L-arginine or equivalent according to the invention may be orally administered or coadministered in a liquid dosage form. For the preparation of solutions or suspensions it is, for example, possible to use water or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulphoxide, triglycerides and the like.

In the case of drinkable solutions the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), gum acacia or other suspension agents selected from the hydrocolloids may also be used.

It is also possible to add preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

Furthermore, controlled release L-arginine according to the invention may be administered separately, or may coadministered with other inventive controlled release biological equivalents or other therapeutic agents. Coadministration in the context of this invention is defined to mean the administration of more than one therapeutic in the course of a coordinated treatment to achieve an improved clinical outcome. Such coadministration may also be coextensive, that is, occurring during overlapping periods of time.

Preferred concurrently administered compounds would be selected from the anti-oxidants, and may include; vitamin E, selenium, beta carotene, vitamin C, α-lipoic acid, tocotrienols, N-acetylcysteine, co-enzyme Q-10, Pycnogenol® (French maritime pine bark extract, Henkel, Inc.), extracts of rosemary such as carnosol, botanical anti-oxidants such as green tea polyphenols, grape seed extract, resveratrol, ginkgo biloba, and garlic extracts. Folic acid may also be added as the preferred vitamin.

The L-arginine of the invention can be incorporated into any one of the aforementioned controlled released dosage forms, or other conventional dosage forms. The amount of L-arginine contained in each dose can be adjusted, to meet the needs of the individual patient, and the indication. One of skill in the art will readily recognize how to adjust the level of L-arginine and the release rates in a controlled release formulation, in order to optimize delivery of L-arginine and its bioavailability. In a preferable embodiment, the amount of L-arginine in a dose ranges from about 500 mg. to about 30 grams. In a more preferable embodiment, the amount of L-arginine in a dose ranges from about 1 grams to about 10 grams. In a still more preferable embodiment, the amount of L-arginine in a dose is about 5 grams. The rate of release would be from 1 hour to 24 hours, with the preferred rate of release being one that does not produce bowel intolerance. Preferred controlled-release formulations would deliver not more than about 3 grams of L-arginine per hour.

Indications treatable using the invention include immunomodulation; protection of the liver and kidneys; cardiovascular disease; liver diseases; arthritis; increased exercise capacity in older subjects; HIV infection; viral replication; tumor reduction; erectile dysfunction: inflammatory bowel disease, and ulcerative colitis. Additional indications treatable using this invention include, but are not limited to, inflammatory, degenerative articular and extra-articular rheumatic disorders, non-rheumatic states of inflammation and swelling, arthrosis deformans, chondropathies, periarthritis, neurodermitis and psoriasis, alcoholic, hepatic and uraemic origin, degeneration of the liver parenchyma, hepatitis, fatty liver and fatty cirrhosis as well as chronic liver disorders, bronchial asthma, sarcoidosis, and ARDS (acute respiratory distress syndrome).

L-arginine and the anti-oxidants have been disclosed as being useful in the treatment of the above indications. The controlled release formulations of the present invention also have utility in the treatment of these indications.

Dosages of the controlled release formulations of the present invention for treatment of these indications may be optimized by one of skill, using conventional dosing trials.

Additionally, controlled release L-arginine formulations according to this invention may have improved effect versus immediate release formulations. These effects include improved bioavailability (AUC); prolonged mean residence time (MRT) in blood; better absorption, higher concentration (C max), and changing the conversion of L-arginine metabolites, such as L-citrulline and nitric oxide with respect to one another. These improvements relate to the more efficient production of NO by L-arginine and its biological equivalents when given in oral formulations.

It will be apparent to those skilled in the art that various modifications and variations can be made in the compositions, kits, and methods of the present invention without departing from the spirit or scope of the invention. Thus, it is intended that the present invention cover the modifications and variations of this invention provided they come within the scope of the appended claims and their equivalents. Additionally, the following examples are appended for the purpose of illustrating the claimed invention, and should not be construed so as to limit the scope of the claimed invention.

### EXAMPLES

### Example 1

A fluid bed granulator (MP-1, Niro Inc. Columbia Md.) equipped with a 16-liter stainless steel container, a pneumatic operator's panel, and a standard design PACF exhaust filter with a nominal rating of 5-20 microns was employed. The bowl used an 8% distribution plate covered with a 100-mesh woven screen. The nozzle used was a Schlick 970, with a 1.2-mm insert, positioned at the lower port of the bowl. A peristaltic pump, equipped with a silicone tubing, was used to deliver the coating solution which was a mixture of Surelease® ethylcellulose and Opadry® hydroxypropylmethylcellulose (HPMC) (Colorcon, West Point Pa.).

Material Description:

| | | |
|---|---|---|
| Solids (core): | L-arginine | 1000.0 g |
| Coating solution: | Surelease ® ethylcellulose | 800.0 g |
| | Opadry ® HPMC | 50.0 g |
| | Deionized water | 816.7 g |

The HPMC was first dissolved in eater and the solution was allowed to deaerate for 30 minutes. The ethylcellulose was then added and mixed for at least 5 minutes with gentile agitation to avoid froth formation. The MP-1 fluid bed was pre-heated without load. The L-arginine powder was then charged to the bowl and fluidized. Pre-heating was done at 50 CMH for 3 minutes and spraying of the solution was started thereafter at 70 CMH. The inlet temperature was set at 60° C. Blowback was set at 20 second interval and the atomization air pressure was kept constant at 2.0 bars. The airflow was raised to and maintained at 85 CMH until conclusion of spraying. Intermittent spraying and drying was performed. Drying was started at 50 CMH with an inlet temperature of 60° C. but was reduced to 50° C. to keep a low product temperature. Drying time was 60 minutes.

The final product consisted of small coated granules of 80% L-arginine and 20% coating. The coating provided taste masking of the bitter L-arginine and produced a sustained-release, powdered drink mix. Additional flavoring and sweetening agents can be added to produce a pleasant tasting powder that can be stirred in water, juice or other beverages, without dose dumping the L-arginine and producing bowel intolerance. The sustained-release L-arginine also results in better absorption and therefore more effective production of nitric oxide. The gradual release of the L-arginine in the gastrointestinal tract does not overwhelm or saturate the absorptive process, and can be presented as substrate for nitric oxide production in an improved manner.

### Example 2

In a first step, L-arginine free base is screened to a particle size range of 150 to 450 microns. The L-arginine is then added to a Glatt (Ramsey, N.J.) fluid bed granulator. The L-arginine particles become the cores for a coated particle. The cores are coated with a 30% w/w aqueous dispersion of EUDRAGIT® (NE30 D, methacrylic acid ester) and talc. This yields coated particles with a dried coating weight equal to about 10% of the total weight of the coated particle. The inlet air temperature is kept at a temperature of 25° C. After drying, the coated particles are screened using a 40 mesh screen.

The resulting, free-flowing particles are then blended and directly compressed using a tableting press according to the following formula:

| | |
|---|---|
| L-arginine, coated particles | 71% |
| METHOCEL ® K100 | 5% |
| (methylcellulose) | |
| Guar Gum (Supercol G-3) | 15% |
| Microcrystalline cellulose | 5% |
| Stearic Acid | 3% |
| Micronized silica | 0.5% |
| Magnesium Stearate | 0.5% |

The resulting tablet is a sustained release formulation that is compressed into a 1,200 mg tablet containing about 732 mg. of L-arginine per tablet.

### Example 3

In a first step, L-arginine is screened to a particle size range of 150 to 450 microns. The L-arginine is then added to a Glatt (Ramsey, N.J.) fluid bed granulator. The L-arginine particles become the cores for a coated particle. EUDRAGIT® (L/S 100, methacrylic acid ester) is dissolved in isopropyl alcohol to form a 15% w/w solution. Triethyl citrate, talc, and water are additionally added to the solution. Total solids content of the resulting mixture is 9.6% w/w. This yields coated particles with a dried coating weight equal to about 10% of the total weight of the coated particle. The inlet air temperature is kept at a temperature of 25° C. After drying, the coated particles are screened using a 40 mesh screen.

The resulting, free-flowing particles are then blended and directly compressed using a tableting press according to the following formula:

| | |
|---|---|
| L-arginine, enteric coated particles | 71% |
| METHOCEL ® K100 (methylcellulose) | 20% |
| Microcrystalline cellulose | 5% |
| Stearic Acid | 3% |
| Micronized silica | 0.5% |
| Magnesium Stearate | 0.5% |

The resulting tablet with enteric coated L-arginine spheres, is delivered to the small intestine where it is gradually released.

### Example 4

A preblend of 98% w/w CARBOPOL® 934 (B.F. Goodrich Chemical, lightly cross-linked acrylic acid allyl sucrose copolymer) and 2% w/w micronized silica is prepared. To this mixture, L-arginine, METHOCEL® K100, stearic acid, and lactose are added according to the following formula:

| | |
|---|---|
| L-arginine | 64.5% |
| CARBOPOL ® 934/silica preblend | 10% |
| METHOCEL ® K100 | 10% |
| Microcrystalline cellulose | 5% |
| stearic acid | 5% |
| lactose | 5% |
| Magnesium stearate | 0.5% |

The resulting mixture is tableted using a direct compression tableting press to form a bioadhesive hydrogel formulation.

### Example 5

A preblend of 98% w/w L-arginine and 2% w/w CAB-O-SIL® micronized silica is formed. To this mixture is added guar gum (AQUALON® G-3), polyvinylpyrrolidone (PVP), calcium carbonate, stearic acid, lactose, and magnesium stearate in the following amounts:

| | |
|---|---|
| L-arginine/CAB-O-SIL ® blend | 49.5% |
| guar gum (AQUALON ® G-3) | 30% |
| polyvinylpyrrolidone (PVP) | 5% |
| calcium carbonate | 5% |
| stearic acid | 5% |
| microcrystalline cellulose | 5% |
| magnesium stearate | 0.5% |

The resulting mixture is tableted using a direct compression tableting press to form a sustained release caplet formulation.

### Example 6

### Clinical Study

Proposed Study Methods

Study subjects. 6 male subjects with an average age of 60 with diagnosed erectile impotence due to cardiovascular disease are recruited. All subjects are to sign a consent form approved by the institutions review board for research involving human subjects. At entry the study subjects will undergo a complete medical evaluation including physical examination, electrocardiogram, blood chemistry, hematology and urinalyses. The exclusion criteria will include any skin disease, active sunburn, significant test abnormality or any active illness.

Study Protocol. Two way cross-over, randomized, double blind, placebo controlled study. The subjects are randomized to receive orally either a controlled release, sweetened and flavored, L-arginine drink mix in a pre-measured amount that yields 5 grams of L-arginine, or a placebo that is sweetened and flavored in the same way. Both treatments are packaged in identical coded packets or sachets so as to be blinded from both the patient as well as the clinician. The subjects are instructed to consume the contents of each packet in a full 8 to 10 oz. glass of water 4 hours before measuring blood flow and sexual stimulation.

The study is repeated using the protocol described above in the same subjects after 1 week of washout, with the order reversed according to code. During the study duration the subjects will be asked to refrain from alcohol intake. Cigarette smoking or intake of caffeinated products are not allowed at least 2 hours before and during the testing.

Vasodilatation. Skin blood flow and temperature in the genital area are determined by laser doppler flowmetry (LDF) technique using a DRT4 Laser Doppler Perfusion and Temperature Monitor (Moor Instruments, Millwey, Devon, England). Standard right angle laser probes (Laser diode 780 nm, output 1 mW, temperature resolution 0.1 C.) are attached to the skin. Data is collected on an IBM Personal Computer using the DRTSOFT software. Monitoring is conducted before and during sexual stimulation. Conditions of room temperature (20° C.), air convection and humidity are kept constant throughout the study.

Results. A significant difference between the placebo and the controlled release L-arginine is measured quantitatively by the Laser Doppler Perfusion technique. The changes in Doppler Flux shift and area under the Flux-time curve (AUC) are most noticeable, with the most marked effect in the AUC of the L-arginine group, because this variable reflects both intensity and duration of vasodilatation. Controlled release L-arginine increased the mean AUC in the flux time curve by a statistically significant amount compared to placebo. The L-arginine group also experienced a marked improvement in ability to achieve erections and the duration of rigidity.

While particular embodiments of the invention have been described in detail, it will be apparent to those skilled in the art that these embodiments are exemplary rather than limiting, and the true scope of the invention is that defined by the following claims.

What is claimed is:

1. A controlled release pharmaceutical composition comprising an agent which enhances or modulates the production of endogenous nitric oxide (NO) in a mammal.

2. The controlled release pharmaceutical composition of claim 1, wherein the endogenous nitric oxide enhancing agent is L-arginine, L-ornithine, L-citrulline, their salts, or complexes.

3. The controlled release pharmaceutical composition of claim 2 wherein the nitric oxide enhancing compound is a peptide of L-arginine, L-ornithine, or L-citrulline.

4. The controlled release pharmaceutical composition of claim 1 wherein the agent is a botanical substance that enhances or regulates endogenous production of nitric oxide.

5. The controlled release pharmaceutical composition of claim 4 wherein the botanical substance is a flavonoid or bioflavonoid.

6. The controlled release pharmaceutical composition of claim 4 wherein the botanical substance is garlic, ginkgo biloba, grape seed extract, or French maritime pine bark extract.

7. The controlled release pharmaceutical composition of claim 7, wherein the concentration of L-arginine ranges from about 10 to about 95 weight percent, based on total weight.

8. The controlled release pharmaceutical composition of claim 2, wherein the concentration of L-arginine ranges from 50 to about 80 weight percent, based on total weight.

9. The controlled release pharmaceutical composition of claim 1 wherein the composition is in the form suitable for delivery orally, mucosally, nasally, ocularly, transdermally, parenterally, vaginally, rectally, or intrauterine.

10. The controlled release pharmaceutical composition of claim 1, wherein the composition is in a capsule, a tablet, or a powdered drink mix dosage form, or other physical system.

11. The controlled release pharmaceutical composition of claim 10, wherein the dosage form comprises reservoir systems with rate-controlling membranes; reservoir systems without rate-controlling membranes; monolithic systems; materials physically dispersed in non-porous, polymeric, or elastomeric matrices; laminated structures; hydrogels; osmotic pumps; or adsorption onto ion-exchange resins.

12. The controlled release pharmaceutical composition of claim 11, wherein the dosage form comprises polymer matrices that are erodible by hydration, or chemically or biologically in the gastrointestinal tract

13. The controlled release pharmaceutical composition of claim 1, wherein the controlled release pharmaceutical composition comprises a rate-preprogrammed drug delivery system, an activation-modulated drug delivery system, a feedback-regulated drug delivery system, or a site-targeting drug delivery system.

14. The controlled release pharmaceutical composition of claim 1, wherein the controlled release agent comprises algal polysaccharides, chitosan, pectin, glucomannan, guar gum, xanthan gum, gum arabic, gum karaya, locust bean gum, keratin, laminaran, carrageenan, cellulose, modified cellulosic substances such as cellulose ether derivatives; methylcellulose, hydroxypropylmethylcellulose, hydroxypropylcellulose, hydroxyethylcellulose, sodiumcarboxymethylcellulose, carboxymethylcellulose carboxypolymethylene, acrylic resin polymers, polyacrylic acid and homologues, polyethylene glycol, polyethylene oxide, polyhydroxylalkyl methacrylate, polyvinylpyrollidine, polyacrylamide, agar, zein, stearic acid, and gelatin.

15. The controlled release pharmaceutical composition of claim 1, wherein the controlled release pharmaceutical composition comprises an enteric coating.

16. The controlled release pharmaceutical composition of claim 15, wherein the enteric coating comprises hydroxypropyl-methylcellulose phthalate, methacryclic acid-methacrylic acid ester copolymer, polyvinyl acetate-phthalate and cellulose acetate phthalate.

17. The controlled release pharmaceutical composition of claim 1, wherein the controlled release pharmaceutical composition comprises a solid dispersion.

18. The controlled release pharmaceutical composition of claim 18, wherein the solid dispersion comprises a water soluble or a water insoluble carrier.

19. The controlled release pharmaceutical composition of claim 19, wherein the water soluble or water insoluble carrier comprises polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid

20. The composition of claim 2 wherein the range of a single dose of L-arginine is from about 500 mg. to 30 grams.

21. The composition of claim 20 wherein an amount of L-arginine in a single dose amount of the composition ranges from about 1 gram to 10 grams.

22. The composition of claim 21 wherein the amount of L-arginine in a single dose is about 5 grams.

23. The composition of claim 1, wherein the endogenous nitric oxide (NO) generating or modulating agent is present in an amount effective to treat diseases that are impacted by NO.

24. A method of treating disorders impacted by nitric oxide comprising administering to a mammal the composition of claim 1.

25. The method of claim 24 wherein the disorder is erectile dysfunction.
